(19) United States
(12) Patent Application Publication
(10) Pub. No.: US 2006/0270917 A1
(43) Pub. Date: Nov. 30, 2006

(54) Body-worn shell and conforming patient table for reproducibly positioning a patient in an imaging apparatus

(76) Inventor: Manfred Pfeiler, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 11/431,211

(22) Filed: May 10, 2006

(30) Foreign Application Priority Data
May 10, 2005 (DE) 10 2005 021 466.5

(51) Int. Cl. A61B 5/00

(52) U.S. Cl. 600/300

(57) ABSTRACT

To reproducibly position a patient in an imaging device, with regard to the coordinate system thereof, so that such a reproducible positioning of the same patient is additionally possible in another imaging device, so that the images generated with both modalities can be congruently overlaid for an image fusion, a body shell is adapted to the patient, the shell supporting the patient for positioning on the patient table. By shaping of the shell contour to conform to the shape of the contour of the table, the position of the shell on the table is defined. Both imaging devices are equipped with a table of the same shape. The desired adaptation of the respective geometries of the image generation systems is achieved by using shell as a test body, equipped with suitable landmarks that are delectable in the images of both modalities.

### BACKGROUND OF THE INVENTION

1. Field of the Invention

The present invention concerns an arrangement for reproducibly positioning a patient in a medical imaging apparatus, and in particular an arrangement allowing substantially identical positioning of the patient in two different, spatially separated imaging modalities for obtaining images with the different modalities at separated points in time. The present invention also concerns an arrangement that assists in fusion of such images of the same cross-section of a patient obtained at different points of time using spatially-separated different imaging modalities.

2. Description of the Prior Art

Until the beginning of the 1970s, medical diagnostic imaging was almost exclusively accomplished with classic x-ray technology, with radiographs. Ultrasound imaging and imaging with nuclear medicine were still at the beginning of their development. X-ray computed tomography (CT) appeared in the late 1970s, while magnetic resonance tomography (MRT) appeared in the 1980s. CT and MRT provide cross-sectional views that show various properties of the examined tissue. CT shows spatially-dependent properties of the x-ray attenuation of the investigated tissue, while MRT shows properties which are related to the nuclear spin of the atoms present in the tissue, primarily the hydrogen atom. Parallel to the development of CT and MRT, nuclear medicine imaging continued to be developed for cross-sectional image representation. An example is SPECT (single photon emission computerized tomography), wherein an image is produced by measurement of the intensity of gamma radiation or photons, emitted from tissue that has been purposefully enriched with an applied radiopharmaceutical of radiation emitting material. Another nuclear medicine method is PET (positron emission tomography), in which a positron emitting material is placed in a comparable manner in tissue areas, and the emitted positrons combine with an electron in direct proximity to their emission location and thus generate gamma radiation. Imaging with ultrasound has also made significant advances.

The non-nuclear medic methods named above all address different tissue properties or identical tissue properties, but with different sensitivity, specificity and type of representation. Depending on the relevant medical question, usually one of these imaging modalities is preferred, but the application of several modalities can decisively increase the diagnostic information for certain problems.

The nuclear medicine images (SPECT, PET) show intensity distributions in the imaged tissue cross-sections the intensity distributions, depending on the composition of the radiopharmaceutical, can represent healthy or normally functioning tissue such as, for example, the heart muscle on the basis of its metabolism, or can represent diseased tissue on the basis of its pathological metabolism such as, for example, carcinogenic metastases in the liver or in the bones. In the non-nuclear medicine images, clearly represented structures are lacking or can only be intimated, so that a metabolically active tissue represented by an intensity distribution either cannot be classified or can only be insufficiently classified in the morphology of the cross-section of the body. For certain problems it would be of particular advantage to detect the precise location of an intensity distribution within an organ, for which it is desirable to bring the nuclear medicine tomogram and the non-nuclear medicine tomogram into precise congruency. There is the task of image registration, which transforms the locations of the same pixels of tissue or an organ of the body or tissue cross-sectional images obtained with two imaging modalities, so that they have the same local coordinates in a common coordinate system, or by transforming such pixel locations in the coordinate system of the one modality into the coordinate system of the other modality. Such a transformation generally cannot be accomplished with only one exposure and/or positioning, because it must be assumed that in the case of imaging with two modalities, even if the image cross-section is produced at the same height in the positioning of the patient, e.g. by placement of the internal organs the border of the body cross-section, the represented organs still appear differently in the respective images. Therefore, image registration is a complicated procedure.

The image registration procedure is followed by a procedure wherein the two images are combined into an “integrated representation” of both image contents. This operation is known as image fusion. This happens by overlaying both (registered) images. For the purpose of better differentiation, the contents of one image, e.g. the nuclear medical image, are created in color and are overlaid on a black and white CT image. Since images of differing modalities are registered and combined, this is often referred to as multimodal registration and fusion; but it would be more accurate, since only two image modalities are involved, to use the term registration and fusion.

This procedure and its fundamental significance are explained in detail in copending U.S. patent application Ser. No. 11/292,960, filed Dec. 2, 2005. Therein it is noted that the fusion of CT images and PET images is considered to be so essential for diagnostics and their correct registration considered to be so difficult that the mathematical operation of a complex image transformation is circumvented by means of a physical solution: combining CT and PET in one device, so that for the placement of the patient from one imaging mode to the other imaging mode, movement of the patient positioning table is sufficient; thus there is no need for a rearrangement of the patient. The exposures of the various modes are processed in one examination, thus directly after each other, so that even a separate movement of the organs, e.g. due to intestinal peristalsis, can be assumed as non-existent or only slight. Thus it can be assumed that the cross-section images of the two modalities (CT and PET) in one device are of practically the same topology (geometry).

Combined CT/PET systems are already commercially available. A CT/PET device was introduced the middle of 2004 (Siemens: Medical Solutions, electromedica, November 2004, pages 16-21). It should be noted that the above considerations for a CT/PET device apply as well for a CT/SPECT device.

Such a CP/PET apparatus, however, does not solve the registration problem that arises in the case of a patient for whom, after an operation and or in the course of a different treatment, primarily PET images are desired for the follow-up examinations, and it is desired to forego CT exposures at the same time, for reasons of the radiation dose to be applied to the patient as well as for reasons of cost. A similar issue arises when a patient, for whom an examination with CT causes a desire for a further examination with PET, is transferred to another institution which also operates a CT/PET apparatus. In addition, at this time it can be assumed that a still-new dual modality system will not be installed, or are only installed on a limited basis, in radiological institutions. Radiology and nuclear medicine are different and usually only nuclear medical institutions have a permit (license) for handling open radioactive substances, such as e.g. radiopharmaceuticals. In addition, the question as to whether and which compromises are to be made for the use of only one of the two modalities is relevant when both modalities are combined in one apparatus. Compromises not only of a technical nature must be addressed, but also compromises of economic efficiency in clinical operation. Of the two modalities, when only one is in operation at a time, the other cannot be used. Due to the significantly longer examination times for PET compared to the CT this means extensive idleness for the CT part of the device. Overall, it therefore appears probable that the aforementioned dual modality systems will not fundamentally replace one or the other of the two single modality apparatuses.

Thus the problem of optimum image registration and fusion remains.

In the above-cited patent application the use of a registration aid is described. In the simplest case, this is a belt that is placed on the patient, to which cylinder-shaped landmark sensors oriented in the direction of the axis of the patient are mounted. These landmark sensors are detectable in a CT image as well as in a nuclear medicine image (PET, SPECT), wherein their respective central points are in topologically identical locations.

For the belt to be effective, as a landmark carrier, if it is removed by the patient after an examination with CT, and if it must be put on again later and at a different location for an examination with PET or SPECT, it must be located in similar manner on the patient body as to position and fit.

For such reproducible placement of the belt, it is helpful if the belt is constructed similar to a corset or undershirt shaped to the patient body, particularly when the configuration of the belt fastener is designed so that it is unique and therewith recordable for each selected adjustment. Such a reproducible corset-type belt would not only offer the advantage of being able to be better positioned again, and at the same places on the periphery of the body cross-section, but also would provide a support function to assist in the task of bringing the internal organs at the height of the body trunk cross-section that is to be displayed into the configuration that existed in preceding examinations.

In an embodiment described the patent application cited above, a body shell is adapted to the patient as shown in FIG. 1a. This body shell 2 is adapted to the patient's body cross-section 1 shown in FIG. 1a by placing the patient into the preformed but still malleable shell 2. If necessary, a grid or mesh can be incorporated into the not-yet-hardened shell material during the fabrication thereof. This shell 2 is fitted to the patient in a still malleable state and is then allowed to harden (cure) in this state. So that the shell 2 can be removed again, for later repeated use, it is equipped with one or more flexible seams or hinges 3. The aperture slot 4 is locked with a closing mechanism 5, for example a Velcro® fastener system. The shell 2 can be equipped with the landmark sensors 6, the cross-section 7 of which in accordance with FIG. 1b is a cylindrical hollow body, which is how it also appears in the CT image.

The cross-section 8 is formed by the interior of the hollow body. In the case of nuclear medicine imaging, the interiean receives a suitable radiation emitting preparation for the landmarks to be generated (in the following referred to as radioisotope for short) in a container adapted to the interior. This preparation, for example, can be a diluted form of the radiopharmaceutical that is injected into the patient for the examination.

The landmark sensors 6 can be designed as a continuous tube that proceeds in a serpentine configuration over the perimeter of the shell 2, so that its cross-section is depicted as shown in FIG. 1b. The cross-section 8 corresponds to the wall of this tube, which appears in the CT image, and the cross-section 8 of its interior, which is filled with the imaging radioisotope for nuclear medicine imaging.

The shell 2 additionally can be provided with sight holes 9 or hole templates as an aid for position marking on the patient body prior to removal of the body shell and for checking the position after putting the body shell back on.

Thus it can be assumed that CT images produced with the help of this shell 2 and nuclear medicine diagnostic images can be joined together without particular problems in the sense of an image fusion. It can be sufficient, in the case of nuclear medicine repeat examinations for image fusion with computer tomograms of identical body cross-sections, to keep an existing set of CT images available. This has the advantage that costs for new CT examinations and the patient radiation dose associated with the examinations are omitted.

Nuclear medicine repeat examinations, as required follow-up examinations after therapeutic treatments, can serve the purpose of tracking the changes in size, shape and/or location of a pathological condition being treated. The nuclear medicine images supply, on the basis of their ability to represent metabolic representations e.g. in the case of tumors, provide diagnostic options that a CT image does not provide, because changes of the structure to be assessed, such as in the case of tumors, are not yet anatomically detectable. For such a time period of follow-up examinations, this body shell 2 accompanies a patient as a customized component used only for that patient.

The shell 2 does not have to have a predominantly closed wall. This wall alternatively can completely or partially be formed of a mesh or struts, as long as this provides the described necessary properties for the shell 2.

It is now assumed that a patient, after examination by means of computer tomography (CT), a different location to conduct a nuclear medicine examination using PET. It is assumed that in this transfer (e.g. due to a body shell in accordance with FIG. 1 that has been put on and still worn) the cross-section that is to be examined ideally remains unchanged. In spite of this, the repositioning from the one device to another, that is the new patient positioning, will have the result, even in the case of an identical image scale of both systems, that the images of the same patient cross-section generated by means of CT and PET will as a rule be misaligned laterally and in height and can also be distorted. With the registration aid for medical images of the patent application cited above, an image adaptation in the sense of topologically congruent overlaying of the two images, is a comparatively simple task for the image evaluation programs with which the predominantly digital-electronic operating image systems usually are equipped with in advance.

The possibility of adaptation of the PET image to the already present CT image, however, requires preparation in advance, i.e. prior to the nuclear medicine examination, namely the activation of the landmarks 6 by means of filling with the suitable radioisotope. This effort involves the preparation of the radioisotope itself including a dilution thereof, filling the landmarks 6 on or in the shell 2 in accordance with FIG. 1 under observation of practices relevant to radiation-protection.

### SUMMARY OF THE INVENTION

An object of the present invention is to provide an arrangement of the above type that can be used not only for reproducibly positioning a patient in a medical imaging apparatus, but also allows the patient to be reproducibly positioned into different, spatially-separated imaging apparatuses, respectively operating according to two different imaging modalities, for obtaining respective images of the same cross-section of the patient at separated points in time.

A further object of the present invention is to provide such an arrangement that allows such images, obtained at spatially-separated imaging apparatuses operating according to two different modalities, at spatially separated points in time, to be brought into registration with each other so that image fusion of the two images can be more readily accomplished.

The first of the above objects is achieved in accordance with the present invention by a body-worn shell that can be reproducibly applied to (worn by) a patient at always the same position on the patient's body, and wherein the shell has an exterior shell contour at one side of the shell, and the top side of a patient table of a medical imaging apparatus has a conforming contour, so that the patient wearing the shell can be reproducibly positioned on the patient table with a transverse cross-section of the patient, within the shell, always being oriented in the same position relative to the longitudinal axis of the table.

The shell can be detachably connected to the top of the patient table.

When the patient is to be examined using two different medical imaging apparatuses respectively operating according to two different imaging modalities, such as a CT apparatus and a nuclear medicine imaging apparatus, with the apparatuses being spatially separated from each other, the shell can accompany the patient as the patient moves from one imaging apparatus to the other, and the patient table in each imaging apparatus has a table top that conforms to the shell. The aforementioned cross-section of the patient thus can be substantially identically positioned in each of the spatially-separated imaging apparatuses, so that respective images can be obtained with the two imaging apparatuses at spatially separated points in time. Because the respective cross-sectional images are substantially identically oriented in each of the two imaging apparatuses, bringing those images into registration with each other for image fusion is facilitated.

The third of the above objects is accomplished in accordance with the invention by providing a shell of the type described above with landmarks that are detectable with each of the two imaging modalities. These landmarks are visible or detectable in the cross-sectional images obtained with the respective imaging modalities. When the respective images are obtained while the patient is wearing the shell, this further facilitates registration of the images for image fusion. Because the shell itself can be reproducibly positioned in each of the two different imaging apparatuses, however, the shell, by itself, without a patient in the shell, can be used in the respective imaging apparatuses for calibration purposes.

The shell can be worn at any suitable body location, such as around the trunk of the patient or on the cranium of the patient. Where appropriate, the shell can be formed of material that has a sufficient rigidity, so that when the patient wears the shell internal body organs are forced into the same position (in cross-section) every time the shell is applied to the patient.

### BRIEF DESCRIPTION OF THE DRAWINGS

FIGS. 1a and 1b schematically illustrate a belt of the type described in copending application Ser. No. 11/292,960.

FIGS. 2a, 2b and 2c schematically illustrate a body-worn shell on a shape-conforming patient table in accordance with the present invention.

FIGS. 3a, 3b and 3c schematically illustrate a further embodiment of the present invention.

### DESCRIPTION OF THE PREFERRED EMBODIMENTS

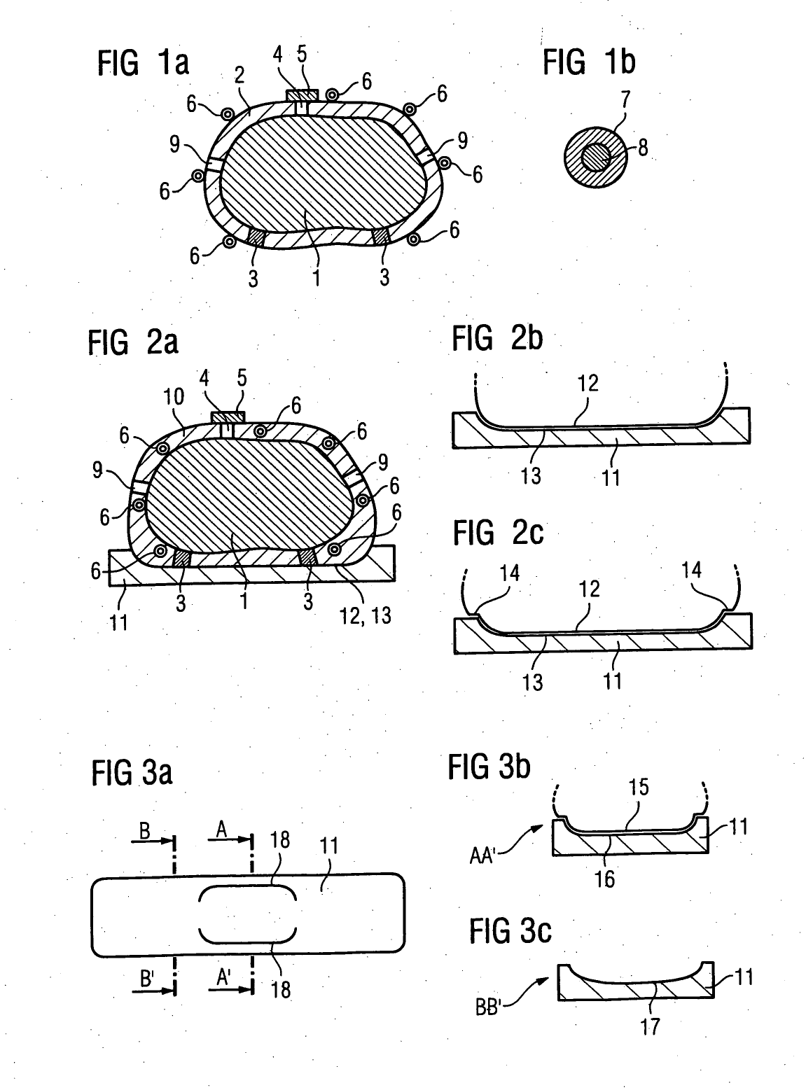

Using the example of the body shell shown in FIGS. 1 and 16, the patient tables of the two imaging devise, such as CT and PET devices, are constructed so that the cross-section of a patient wearing the shell 2 can be reproducibly positioned transversely to the longitudinal axis of the examination equipment. In accordance with the embodiment shown in FIG. 2a, this can be achieved by placing a standard preformed body shell 10 otherwise of the same as the shell 2 on a patient table 11, such that it conforms in a form-locking manner to the table 11. The shape or contour of the top of the patient table 11 is designed so that patients without a body shell 10 can be examined in the normal manner, thus without restriction. For an exposure, a defined or defined producible position of the table 11 with regard to the longitudinal axis of the respective devices is necessary.

The form-locking fitting of the shell 10 with the contour 12 on its lower side into the bed 11 with the contour 13 on the upper side of the patient table 11 is illustrated in FIG. 2b. For better representation a small space is shown between these contours, but in practice they are directly adjacent one another. FIG. 2c shows an incorporated ledge 14 that makes a simpler fitting of the shell 10 into the bed 11, which can be better inspected as to proper positioning.

If the tables 11 of the two imaging devices that are in separate locations allow an identical and reproducible positioning of the patient 1 with the shell 10 adapted to the patient 1, the topology (at a right angle to the longitudinal axis of the devices) of the shell 10 in FIG. 2a with regard to the patient tables 11 in both devices will be identically transferred from one device to the other. All that is now required is an adjustment of the image display of both devices with regard to their orientation within the image plane and with regard to the scale in order to ensure that the images produced with both modalities of the same cross-section of the patient 1 are topologically identical, thus congruent.

The adaptation of both devices to each other that is necessary in this respect can be achieved by placing a suitable sample body, which can be of the shell 10 without patient 1, first into the computed tomography apparatus as one of the two imaging devices and then, using landmarks activated by means of radioisotope, placing the suitable sample body into the other device, namely the PET device. The topology of the image display of both devices is then calibrated to this sample body. It is assumed that the imaging of the devices is sufficiently stable so that a recalibration in the case of normal use of the devices is only occasionally necessary.

If the two devices, in the above example the CT device and the PET device, are prepared so that the images of the cross-section of a patient 1 match in advance due to the shell 10 that has been adapted to and placed on said patient for both modalities, and the images can be congruently overlaid both without nuclear medicine activation of the landmarks 6 and without further image processing for adaptation of the topology. The shell 10 thus no longer needs to be equipped with landmark sensors 6.

Instead of the embodiment described in FIG. 2a involving fitting of the shell 10 worn by the patient into the table 11, other embodiments are also possible which can satisfy the condition that the table 11 does not represent a restriction for examinations of patients without a patient shell 10. For example, the table 11 can be provided with boreholes, into which pins or bolts 3 mounted on shell 2 in FIG. 1a engage. Both embodiments are, with respect to the table 11, suitable for retrofitting, wherein e.g. a retrofitting with regard to forming the contour in the table 11 by means of form pieces that can be attached to the table 11 by means of adhesion.

Although in the above discussion PET is noted a nuclear medicine as an imaging method, SPECT can likewise be used.

Further it is noted that in comparable manner MRT can be used in place of CT, as long as the shell 2 in FIG. 1a or the shell 10 in FIG. 2a is built of materials which, as in the case of the CT also in the case of the MRT disturb the imaging to the least possible extent, and the material used for the landmark sensors 6 results in a sufficiently high contrast with regard to the shell 2 or the shell 10 in the images. Different materials can come into consideration for the two different modalities of CT and MRT.

The question remains of how to use the device of the other modality to find the appropriate, thus identical body cross-section of one of the various cross-section images acquired by one of the two modalities along the longitudinal direction of the patient and thus in the longitudinal direction of the shell 10.

If the shell 10 is equipped with landmark sensors 6, no problem arises if, in the case of a nuclear medicine examination, the sensors 6 were activated with a radioisotope provided for this purpose. Cross-sectional images of the landmark sensors 6 that are not parallel to the longitudinal axis of patient and shell 10, by comparison of the images taken with different modalities with respect to the displayed landmarks 6, allow inference of image pairs which belong together, as also shown and explained in the above-cited patent application. In the patent application, among other things, a tube is specified as an embodiment for the landmark sensors 6, which proceeds along a serpentine path, and that path diagonally to the longitudinal axis of the shell 2 or of the shell 10, on or the path wall thereof. The tube serving as a landmark sensor 6 alternatively can be wrapped around the longitudinal axis of the shell 10, outside or inside its wall. In the CT image or in the nuclear medicine diagnostic image the tube serving as a landmark sensor 6 then would not be shown in full cross-section, but rather is shown cut at an angle. The position of the sectional image depends on the location along the longitudinal axis. In a CT image or in a nuclear medicine diagnostic image several parallel tube windings would result in several diagonal cuts displayed in the image, which can facilitate the registration, thus the production of topological congruence, of the images of different modalities.

The following explains how matching cross-section images can be found from both modalities by the longitudinal direction of the patient and thus in the longitudinal direction of the shell 10, for the case wherein the shell 10 is not provided with landmark sensors 6. For a skilled radiologist it may not constitute a problem if even a certain effort were necessary to find locally matching image pairs from a series of CT images and a series of nuclear medicine diagnostic images of the same examination area, assuming it is ensured by the above described application of the shell 10 that there are matching image pairs, i.e. topologically identical or identical image pairs, exist at all in the supply of images.

It would be a simplification if each image, even in advance, can be assigned a specified position along the longitudinal axis of the shell 10. This would be possible if the shell 10 is provided with markings along its longitudinal axis which can be moved to using the sighting device of the imaging device currently being used during the examination.

It would be easier for the person performing the examination if the shell 10 could also be placed reproducibly on the table 11 with reference to the longitudinal axis of the table 11, wherein it can be assumed that the imaging devices being used can move in a defined manner to the longitudinal positions of their tables. In addition to a reproducible placement of the shell 10 on the table 11 also in longitudinal direction an embodiment in accordance with FIG. 3 is also conceivable for the shell 10 and the table 11. The shell 10 on its underside has the form of the profile 15 in accordance with the section AA′ shown in FIG. 3b, so that with regard to the cross section it fits form-locking into the corresponding contour with the profile 16 of the table 11. This also corresponds to the representation in FIG. 2a for the transverse direction of the table 11. Edges in the forming of the table 11 as marked by the lines 18 in FIG. 3a provide for reproducible positioning also in the longitudinal direction, the lines 18 representing even edges of the forming in view of the table 11. Outside of this contour the table 11, in accordance with the section BB′ shown in FIG. 3c, on its topside has a more or less flat profile 17 normally provided for patient beds. Accordingly, for a form-locking fitting the contour of the underside of the shell 10 must also correspond in the longitudinal direction to the contour of the bed 11.

Thus, in two spatially separated installed examination devices, a topologically identical patient positioned for the imaging has been achieved so that without or with only slight effort in image processing the images of different modalities can be fused, i.e. overlaid topographically.

The invention also allows the data acquired in the CT imaging to be used for so-called attenuation correction in nuclear medicine imaging.

A brief explanation follows as to the meaning of “attenuation correction”.

In collecting data for a CT exposure the path of an x-ray beam from the x-ray generator to an element of the detector is considered. The x-ray nrsm penetrates the object and is attenuated on its path by the materials located in its path. This attenuation is a measurement for the so-called attenuation coefficients of the penetrated materials and for the paths over which the respective attenuation coefficients are found. Ultimately, the path integral of the attenuation coefficient is obtained along the considered ray path. Using such data for many ray paths, the CT image can be reconstructed by a computer, which then is the image representation of the local distribution of the attenuation coefficient in the considered patient cross-section.

If one analogizes the computed tomography method or nuclear medicine, thus PET and SPECT, a detector element is considered to lie in a straight line of the radiation sources lying on its path and sums the radiation directed thereat, in the sense of a path integral of the (locally distributed) radiation intensities detected thereby. An image reconstruction takes place analogously to the computing operations of CT. The radiation sources are not independent of the surrounding tissue, but are bound to or in the composition of the examination subject. These materials, however attenuate rays emitted from the radiation sources differently on their paths to the detector element, depending on the location of the radiation source and depending on the kind and extent of the radiating or non-radiating materials that are passed through. For this reason signals reach the detector element that depend on the radiation sources located in the object as well as the distribution of the attenuation coefficient in accordance with the materials in the object. With these radiation signals alone only an imperfect image reconstruction would be possible. The reconstruction result can be improved by assumptions about the attenuation coefficient distribution, which are incorporated in correctional algorithms or by additionally equipping specified designs of nuclear medicine imaging devices with a radiation measurement device that supports the estimation of the attenuation coefficient distribution.

It would be particularly favorable if the actual distribution of the attenuation coefficients were available, as in a CT image. The opportunity exists to convert a (x-ray) CT image of the cross-section of a patient of whom a nuclear medicine image is to be obtained, into an image for such an isotope radiation and use it for correction of the radiation attenuation, attenuation correction for short. An advantage of the above-mentioned combined devices with identical topologies of their two image modalities, is that after creation of a CT image, the CT data can be used immediately for the attenuation correction of the subsequently-to-be-created nuclear medicine images, in order to be available for that purpose already during the nuclear medicine data acquisition.

In the case of separated devices, even with an existing CT image the acquired data could not be used, or could be used only conditionally, for a nuclear medicine exposure at a later time in a different place for positioning of the patient for an attenuation correction, because in general the topologies of the devices do not correspond to each other nor with respect to an instantaneous body cross-section of the patient.

The above-described invention for reproducible patient positioning as a requirement for the topological adaptation of the devices of different modalities located at different areas allows attenuation correction, by also allowing attenuation values of the nuclear medicine imaging to be supplied to the CT images or their data. Such a procedure would be in particularly useful for device pairs which are installed in the same institution but not at the same location, or for devices which come from the same manufacturer.

The considerations employed for the shell 2 in FIG. 1a and the shell 10 in FIGS. 2a and 3a can also be used for other body parts, such as the cranium. In all cases the issue is whether one standard shell or a very few standard shells is/are needed, as a result of which there would be no need for individual adaptation.

Although modifications and changes may be suggested by those skilled in the art, it is the intention of the inventor to embody within the patent warranted hereon all changes and modifications as reasonably and properly come within the scope of his or her contribution to the art.

I claim as my invention:

1. A medical imaging system comprising:
a medical imaging apparatus having a patient table adapted to support a patient in the medical imaging apparatus, said patient table having a table top and a longitudinal axis;
a shell adapted to be reproducibly applied to a patient at separated points in time at a substantially identical position on the patient at each of the separated points in time, said shell having an exterior side that is adjacent said table top when the patient wearing the shell is placed on the table top, said exterior side of said shell having a shell contour; and
said table top having a table top contour conforming to said shell contour that causes said shell to assume a substantially identical orientation on said patient table at said separated points in time in a plane substantially transverse to said longitudinal axis.

2. A medical imaging system as claimed in claim 1 wherein said medical imaging apparatus is a first medical imaging apparatus and wherein said patient table is a first patient table, and wherein said medical imaging system further comprises:
a second medical imaging apparatus, spatially separated from said first medical imaging apparatus, said second medical imaging apparatus having a second patient table adapted to receive a patient thereon, said second patient table having a table top and a longitudinal axis, said second table top having a table top contour therein substantially identical to the table top contour of the first patient table, causing said shell to be placed on said second patient table with said plane in a substantially identical orientation as on said first patient table.

3. A medical imaging system as claimed in claim 1 wherein said shell has a shape allowing said shell to be worn around a trunk of the patient.

4. A medical imaging system as claimed in claim 3 wherein said shell is comprised of material having a sufficient rigidity to maintain organs within the trunk of the patient in a substantially identical position, in said plane, at said separated points in time.

5. A medical imaging system comprising:
a medical imaging apparatus having a patient table adapted to support a patient in the medical imaging apparatus, said patient table having a table top and a longitudinal axis;
a shell adapted to be reproducibly applied to a patient at separated points in time at a substantially identical position on the patient at each of the separated points in time, said shell having an exterior side that is adjacent said table top when the patient wearing the shell is placed on the table top; and
a releasable fastening arrangement for temporarily affixing said exterior side of said shell to said table top of said patient table at one and only one orientation relative to said longitudinal axis.
